(19) United States
(12) Patent Application Publication
(10) Pub. No.: US 2019/0076118 A1
(43) Pub. Date: Mar. 14, 2019

(54) Product Warming Device

(71) Applicant: Wiesman Holdings, LLC, York, PA (US)

(72) Inventor: Ryan Dohm, York, PA (US)

(73) Assignee: Wiesman Holdings, LLC, York, PA (US)

(21) Appl. No.: 15/702,143

(22) Filed: Sep. 12, 2017

(51) Int. Cl. A61B 8/00

(52) U.S. Cl. CPC A61B 8/4281; A61B 8/44; A61M 2205/36; A61M 2205/058; A61B 8/546

(57) ABSTRACT

A product warming device comprises an outer housing, a temperature regulating device disposed within the outer housing, and a heating system disposed within the outer housing. The temperature regulating device includes a first warming assembly defining a first package receiving space and a second warming assembly defining a second package receiving space. A first warming body of the first warming assembly has an outer wall recessed section expanding an upper opening of the first package receiving space. The heating system is adapted to heat the first warming assembly and the second warming assembly to a temperature set point.

### FIELD OF THE INVENTION

The invention relates to a warming device and, more particularly, to a product warming device having a pair of warming assemblies.

### BACKGROUND

Ultrasound diagnostic procedures are used in assessing and diagnosing a wide variety of medical conditions related to internal organs. These procedures are widely used in obstetrics and cardiology, among other medical specialties.

An ultrasound coupling agent, otherwise known as a “gel”, is designed to act as a conductive barrier between an ultrasound probe and the outer layer of skin, known as the epidermis. The application of a gel to the patient's body lubricates the skin and prevents the sound waves from being trapped or reflected by air pockets that might distort the image and lead to an incorrect diagnosis.

The gel is typically warmed to make a patient much more comfortable during the procedures using the gel. This is commonly performed using known gel warmers, such as a single bottle and multi-bottle gel warmer that rapidly heats the gel and consistently monitors temperature.

However, most known ultrasound coupling flexible pouches are rigid bottle and interfere with storage, transport, and the optimal application of coupling gel. While flexible packages, or pouches, are becoming more known, there is need for a warming device that can consistently warming varying sized packages, such as varied sized pouches.

### SUMMARY

A product warming device according to the invention comprises an outer housing, a temperature regulating device disposed within the outer housing, and a heating system disposed within the outer housing. The temperature regulating device includes a first warming assembly defining a first package receiving space and a second warming assembly defining a second package receiving space. A first warming body of the first warming assembly has an outer wall recessed section expanding an upper opening of the first package receiving space. The heating system is adapted to heat the first warming assembly and the second warming assembly to a temperature set point.

### BRIEF DESCRIPTION OF THE DRAWINGS

These and other features, aspects, and advantages of the invention will become better understood with regard to the following description, appended claims, and accompanying drawings which form a part of the specification and in which like numerals are used to identify like parts throughout and where:

FIG. 1 is a perspective view of a product warming device according to the invention, showing containers about to be positioned there within;

FIG. 2 is a perspective view of a product warming device according to the invention, showing containers positioned there within;

FIG. 3 is a rear perspective exploded view of the product warming device of FIG. 2;

FIG. 4 is a front perspective exploded view of the product warming device of FIG. 2, showing a temperature regulating device according to the invention;

FIG. 5 is front perspective exploded view of the temperature regulating device of FIG. 4;

FIG. 6 is side perspective exploded view of the temperature regulating device of FIG. 4;

FIG. 7 is an exploded view of a heating system of the product warming device according to the invention;

FIG. 8 is a section view of the product warming device of FIG. 2 taken along line 8-8; and

FIG. 9 is a section view of the product warming device of FIG. 2 taken along line 9-9.

### DETAILED DESCRIPTION OF THE EMBODIMENTS

Exemplary embodiments of the present invention will be described hereinafter in detail with reference to the attached drawings, wherein like reference numerals refer to like elements. The present invention may, however, be embodied in many different forms and should not be construed as being limited to the embodiments set forth herein; rather, these embodiments are provided so that the present disclosure will be thorough and complete, and will fully convey the concept of the disclosure to those skilled in the art.

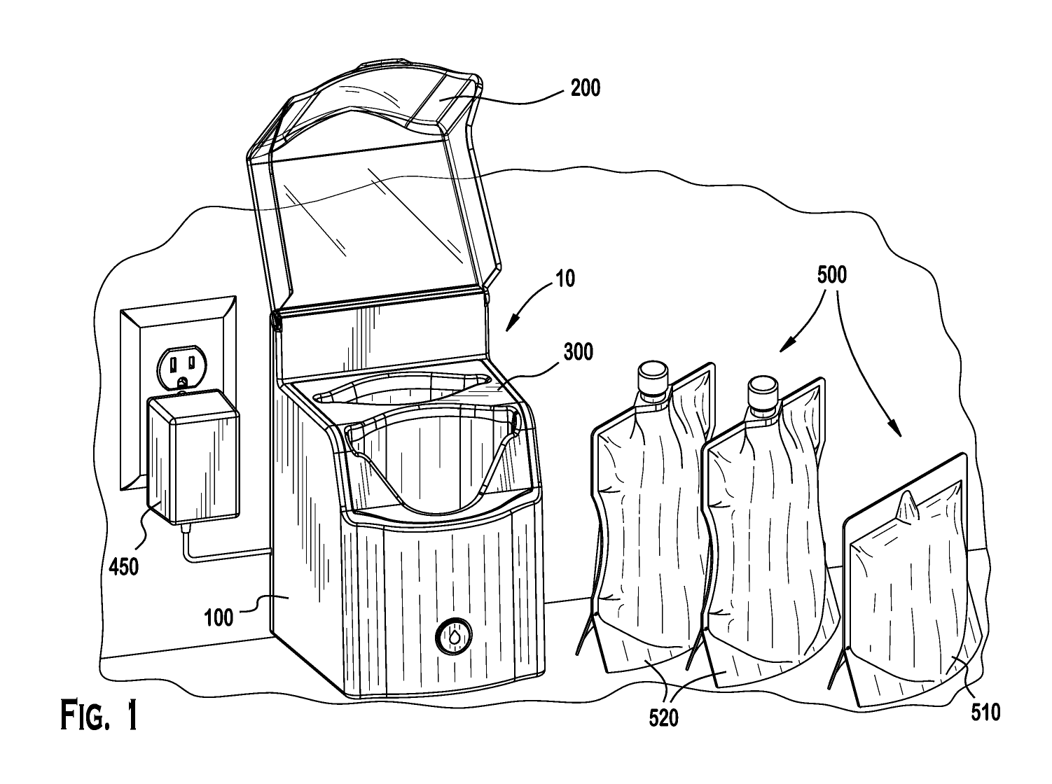

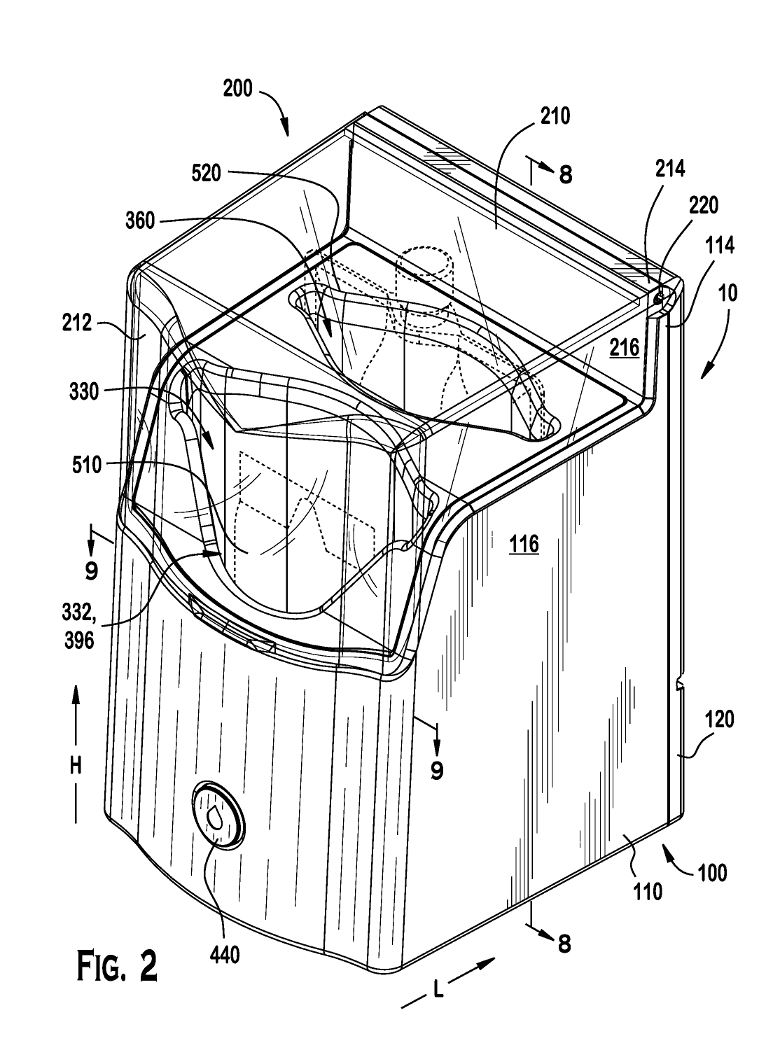

A product warming device 10 according to the invention is shown generally in FIGS. 1 and 2. The product warming device 10 includes an outer housing 100, a lid 200 pivotally attached to the outer housing 100, a temperature regulating device 300 disposed within the outer housing 100, and a heating system 400 disposed within the outer housing 100.

The outer housing 100, as shown in FIGS. 2-4, includes a base housing 110 and a back cover 120. The outer housing 100 is formed of a plastic material.

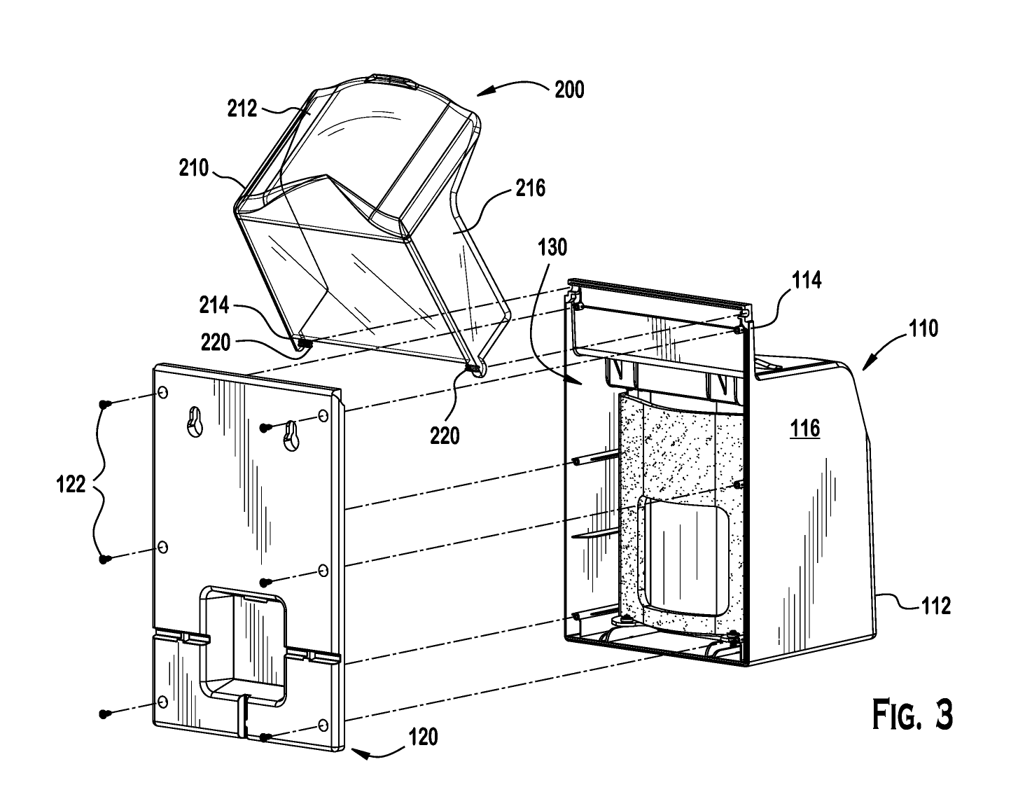

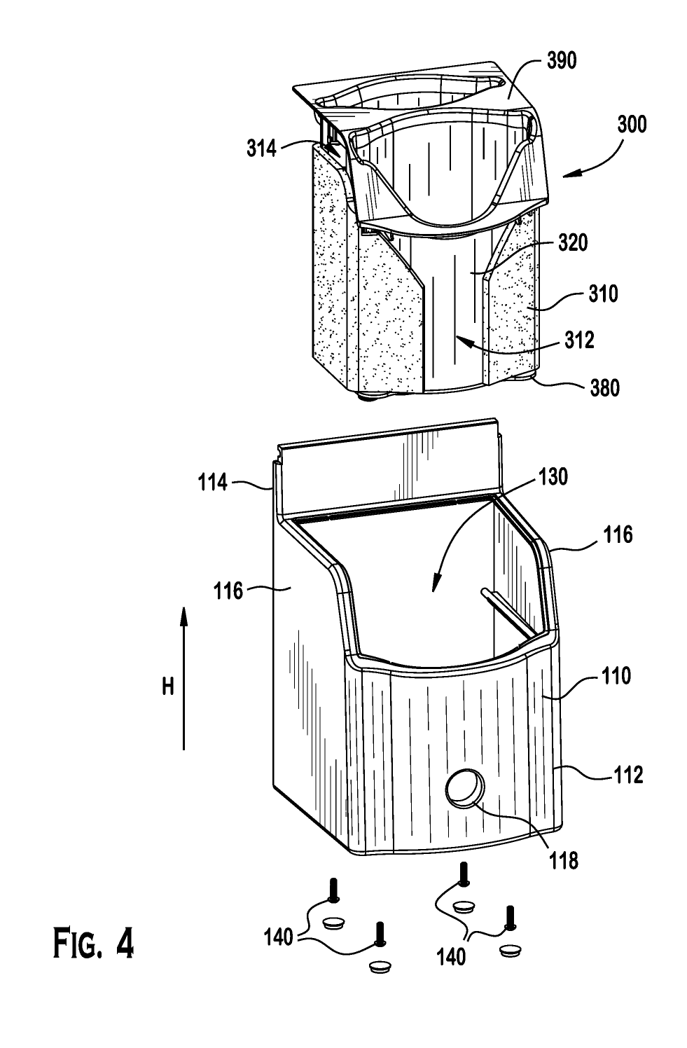

The base housing 110, as shown in FIGS. 3 and 4, at least partially defines a temperature regulating device receiving space 130 between a front side 112, a back side 114, and opposite sidewalls 116 of the base housing 110 connecting the front side 112 to the back side 114. An actuation device receiving space 118 extends through an approximate center of the front side 112. The front side 112 has a front height in a height direction H less than a back height of the back side 114. In the shown embodiment, the sidewalls 116 have an intermediate height in the height direction H greater than the front height and less than the back height.

The back cover 120, as shown in FIG. 3, is a substantially planar member having a cable plug receiving passageway 122. The back cover 120 is attached to the back side 114 of the base housing 110 by a plurality of fasteners 122 to enclose the temperature regulating device receiving space 130. In the shown embodiment, the fasteners 122 are screws. In other embodiments, the fasteners 122 may be a plurality of latches, an adhesive, or any other type of fastener known to those with ordinary skill in the art.

The lid 200, as shown in FIGS. 1-3, has a lid body 210 formed of a transparent plastic material and a hinge device 220. The lid body 210 has a front side 212, a back side 214, and opposite sidewalls 216 extending between the front side 212 and the back side 214. As shown in FIG. 2, the front side 212 has a front height in the height direction H greater than a back height of the back side 214. In the shown embodiment, the sidewalls 216 have an intermediate height in the height direction H greater than the front height and less than the back height. The hinge device 220 is attached to the back side 214 of the lid body 210.

The temperature regulating device 300 is shown in FIGS. 4-6 and includes an inner housing 310, a first warming assembly 320 and a second warming assembly 350. The first warming assembly 320 and the second warming assembly 350 are connected and both disposed within the inner housing 310, a warming base 380 disposed below the first warming assembly 320 and the second warming assembly 350, and a cover housing 390 disposed above the first warming assembly 320 and the second warming assembly 350.

The inner housing 310, as shown in FIG. 4, has a front opening 312 and extends continuously along three other perpendicular sides to define a warming assembly receiving space 314. The warming assembly receiving space 314 extends through the inner housing 310 in the height direction H. In the embodiment shown in FIG. 4, the inner housing 310 tapers toward the front opening 312 in the height direction H.

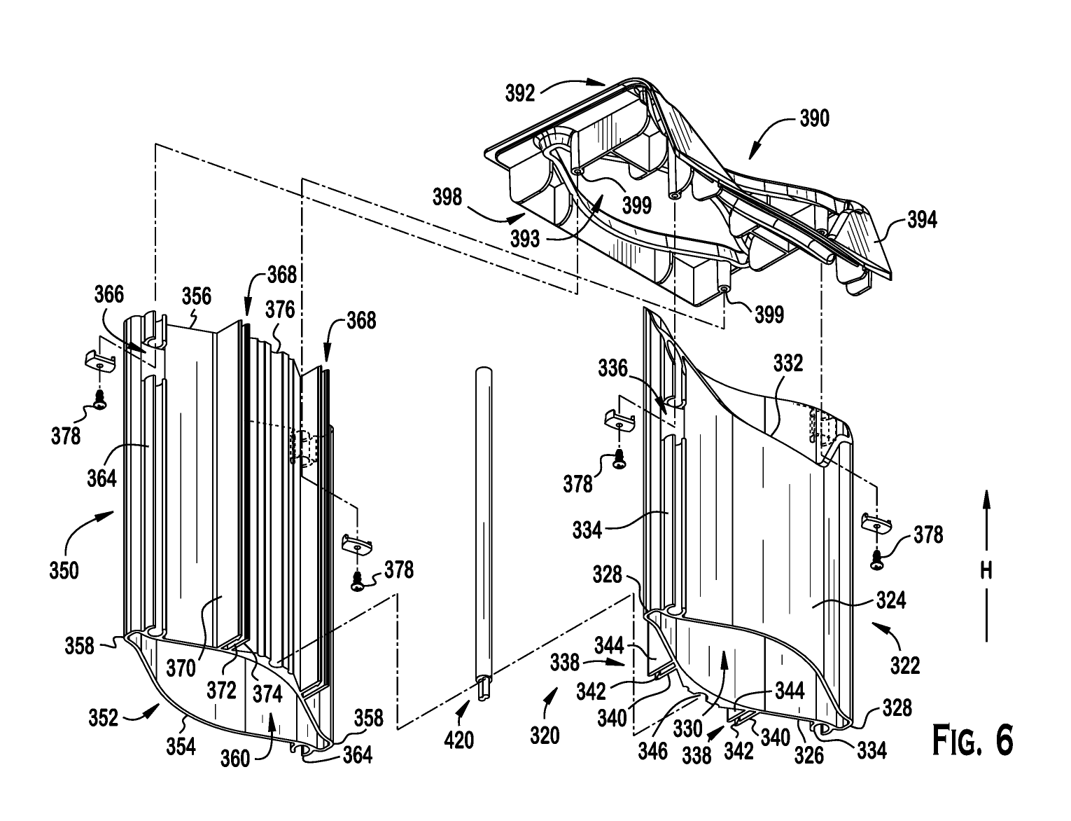

The first warming assembly 320, as shown in FIG. 6, includes a first warming body 322, a plurality of first fastener receiving columns 334, a plurality of first connection devices 338, and a first heating element receiving section 346. In the shown embodiment, the first warming assembly 320 is monolithically formed of a thermally conductive metal material.

The first warming body 322, as shown in FIG. 6, includes a first outer wall 324 and a first inner wall 326 connected by a pair of first warming sidewalls 328. In the shown embodiment, the first outer wall 324 and the first inner wall 326 are both semi-circular shaped members having an elongated body. The first outer wall 324, the first inner wall 326, and the first warming sidewalls 328 together define a first product receiving space 330. The first outer wall 324 has an outer wall recessed section 332 disposed on an upper edge of the first outer wall 324. The outer wall recessed section 332, as shown in FIG. 6, has a highest point in the height direction H at the positions at which the first outer wall 324 is connected to each of the first warming sidewall 328 and a lowest point in the height direction H at an approximate midpoint between the first warming sidewalls 328. The outer wall recessed section 332 expands an upper opening of the first product receiving space 330.

One first fastener receiving column 334, as shown in FIG. 6, is disposed on each of the first outer wall 324 and the first inner wall 326. Each first fastener receiving column 334 extends continuously along the walls 324, 326 in the height direction H aside from an interruption at a column opening 336. In the shown embodiment, the column opening 336 is disposed adjacent an upper edge of the first warming body 322.

The first connection devices 338, as shown in FIG. 6, are disposed on the first inner wall 326. The first connection devices 338 each include a first locking arm 340 and a first guide arm 344 connected to and extending away from the first inner wall 326. The first locking arm 340 and the first guide arm 344 extend parallel to one another. A first latch 342 is disposed on an end of the first locking arm 340 opposite the first inner wall 326 and extends into a space between the first locking arm 340 and the first guide arm 344. In the shown embodiment, two first connection devices 338 extend from the first inner wall 326 and are positioned symmetrically about a center of the first inner wall 326.

The first heating element receiving section 346, as shown in FIG. 6, is a semi-circular recess extending continuously along the first inner wall 326 in the height direction H. The first heating element receiving section 346 is disposed approximately centrally on the first inner wall 326.

The second warming assembly 350, as shown in FIG. 6, includes a second warming body 352, a plurality of second fastener receiving columns 364, a plurality of second connection devices 368, and a second heating element receiving section 376. In the shown embodiment, the second warming assembly 350 is monolithically formed of a thermally conductive metal material.

The second warming body 352, as shown in FIG. 6, includes a semi-circular shaped second outer wall 354 and a semi-circular shaped second inner wall 356 connected by a pair of second warming sidewalls 358. The second outer wall 354, the second inner wall 356, and the second warming sidewalls 358 together define a second product receiving space 360.

One second fastener receiving column 364, as shown in FIG. 6, is disposed on each of the second outer wall 354 and the second inner wall 356. Each second fastener receiving column 364 extends continuously along the walls 354, 356 in the height direction H aside from an interruption at a column opening 366. In the shown embodiment, the column opening 366 is disposed adjacent an upper edge of the second warming body 352.

The second connection devices 368, as shown in FIG. 6, are disposed on the second inner wall 356. The second connection devices 368 each include a second locking arm 370 and a second guide arm 374 connected to and extending away from the second inner wall 356. The second locking arm 370 and the second guide arm 374 extend parallel to one another. A second latch 372 is disposed on an end of the second locking arm 370 opposite the second inner wall 356 and extends into a space between the second locking arm 370 and the second guide arm 374. In the shown embodiment, two second connection devices 368 extend from the second inner wall 356 and are positioned symmetrically about a center of the second inner wall 356.

The second heating element receiving section 376, as shown in FIG. 6, is a semi-circular recess extending continuously along the second inner wall 356 in the height direction H. The second heating element receiving section 376 is disposed approximately centrally on the second inner wall 356.

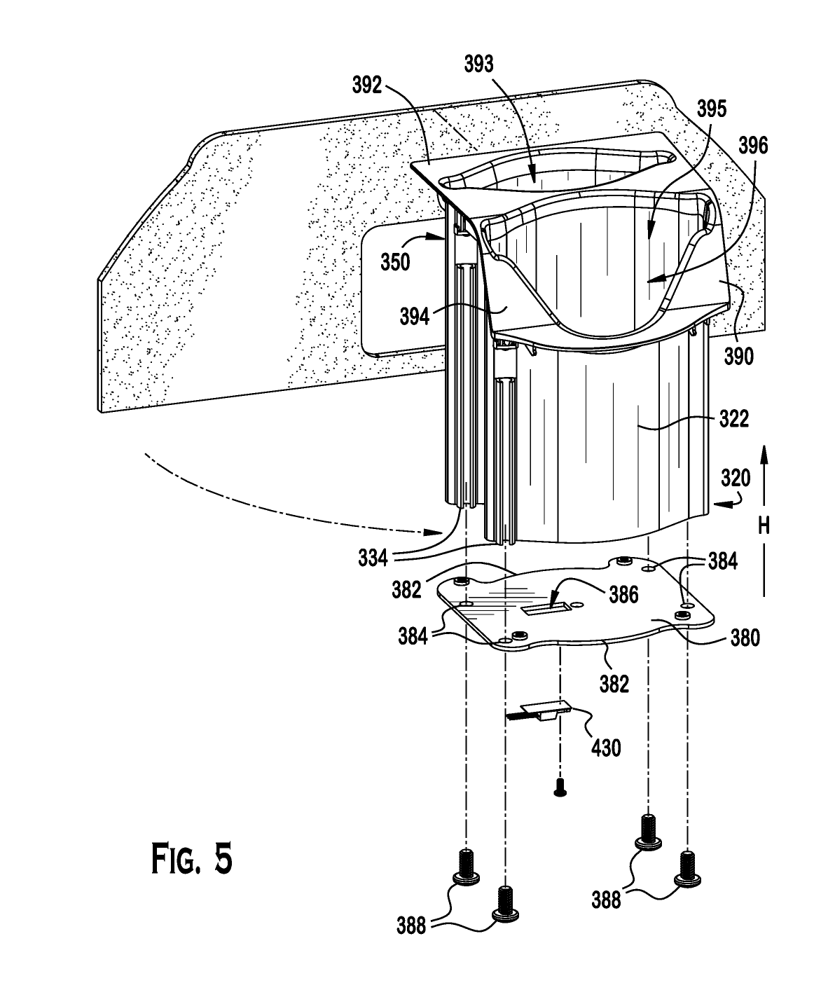

The warming base 380, as shown in FIG. 5, is a planar member having an approximately rectangular shape with opposing semi-circular sides 382. The warming base 380 has a plurality of fastener receiving passageways 384 and a temperature control device receiving space 386 extending through the warming base 380.

The cover housing 390, as shown in FIGS. 5 and 6, includes a top planar surface 392, a front planar surface 394 extending downward at an angle from the top planar surface 392, and a bottom 398. The cover housing 390 is formed of a plastic material. The top planar surface 392 has a first annular opening 393 extending through the top planar surface 392. The front planar surface 394 has a second annular opening 395 extending through the front planar surface 392. The second annular opening 395 includes an outer cover recessed section 396 having a shape complementary to the outer wall recessed section 332 described above. The bottom 398 of the cover housing 390 has a plurality of cover fastener receiving passageways 399.

The heating system 400 is shown in FIGS. 5-7 and includes a controller 410, a heating element 420, a temperature control device 430, an actuation device 440, and a power supply 450.

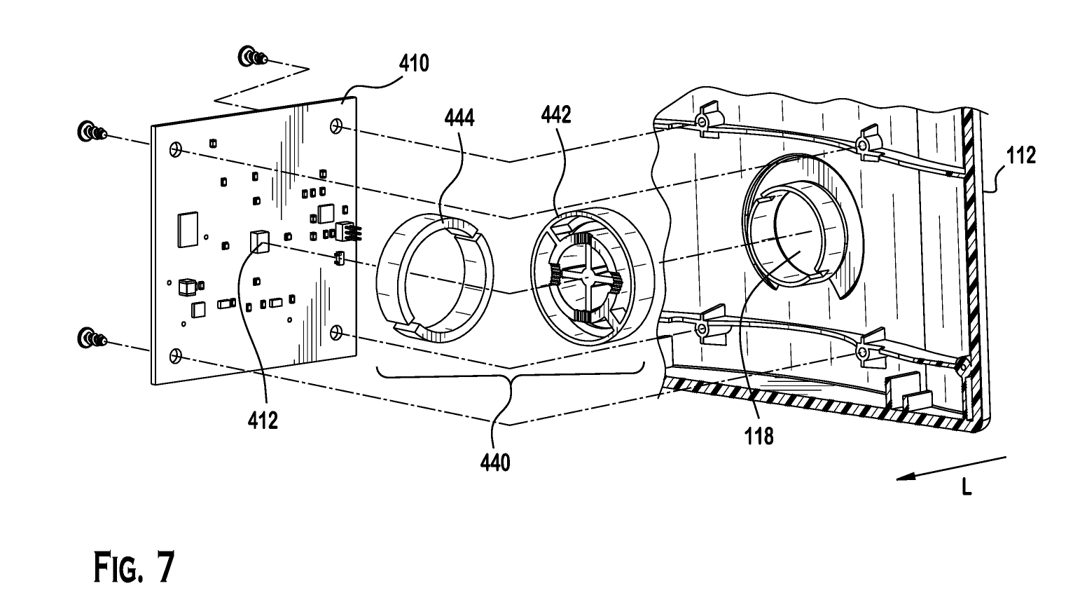

The controller 410, as shown in FIG. 7, comprises a printed circuit board (PCB) having a plurality of electronic components disposed thereon. The controller 410 includes a memory and a processor executing an algorithm stored on the memory; the memory is a non-transitory computer readable medium such as ROM. The controller 410 includes an activation button 412 disposed on the PCB and operable to activate the processor when depressed.

The heating element 420 is shown in FIG. 6. The heating element 420 is an elongated member such as a rod and comprises a material adapted to efficiently transfer heat. In an embodiment, the heating element 420 comprises copper, aluminum, or stainless steel. In other embodiments, the heating element 420 may comprise any material known to efficiently transfer heat.

The temperature control device 430 is shown in FIG. 5. The temperature control device 430 is electrically connected to the controller 410 and is capable of generating heat in response to an electrical signal. The temperature control device 430 is further capable of acting as a thermocouple and outputting an electrical signal indicative of a sensed temperature.

The actuation device 440 is shown in FIG. 7 and includes a switch 442 and a stabilizer ring 444 disposed within the switch 442. The switch 442 is an approximately cylindrical member sized to fit in the actuation device receiving space 118 and be movable within the actuation device receiving space 118 along the longitudinal direction L.

The power supply 450 is shown in FIG. 1. The power supply 450 is electrically connected to the controller 410 and may be any power supply known to those with ordinary skill in the art capable of transmitting power from a known outlet to the controller 410 described herein.

The assembly of the product warming device 10 will now be described in greater detail with reference to FIGS. 1-9.

The lid 200 is connected to the base housing 110 by an attachment of the hinge device 220 with a top of the back side 114 of the base housing 110, as shown in FIGS. 1 and 2. The lid body 210 is pivotable with respect to the base housing 110 about the hinge device 220 between an open position shown in FIG. 1 and a closed position shown in FIG. 2. A shape of the lid body 210 is complementary to the shape of the base housing 110 such that, when the lid 200 is attached to the base housing 110 at the hinge device 220 and is in the closed position shown in FIG. 2, a height of the assembled outer housing 100 and lid 200 in the height direction H is substantially constant in a longitudinal direction L; a combined height of the front side 112 of the base housing 110 and the front side 212 of the lid body 210 is approximately equal to a combined height of the back side 114 of the base housing 110 and the back side 214 of the lid body 210, and approximately equal to a combined height of the sidewalls 116 of the base housing 110 and the sidewalls 216 of the lid body 210.

The temperature regulating device 300 is assembled as shown in FIGS. 4-6.

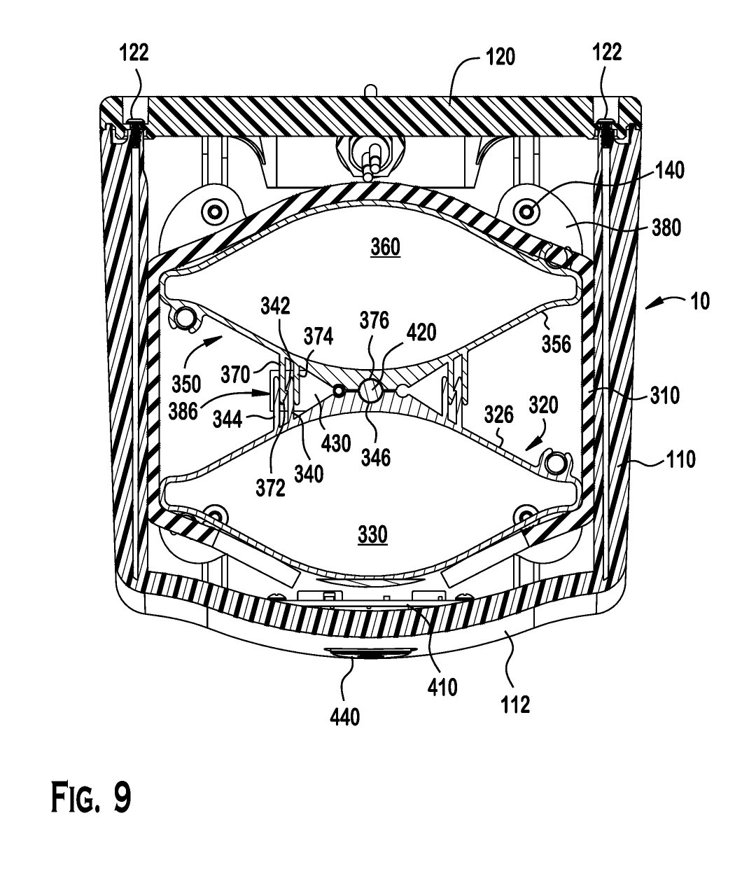

As shown in FIGS. 6 and 9, the first warming assembly 320 is attached to the second warming assembly 350. Each first connection device 338 is connected to one second connection device 368. The connection devices 338, 368 are moved toward one another with the first locking arm 340 moving into the space between the second locking arm 370 and the second guide arm 374 and the second locking arm 370 moving into the space between the first locking arm 340 and the first guide arm 344. In a connected position shown in FIG. 9, the first latch 342 engages with the second latch 372 and the first inner wall 326 abuts the second inner wall 356. The first and second guide arms 344, 374 hold the first and second locking arms 340, 370 between them and maintain the engagement of the first latch 342 with the second latch 372. The heating element 420, as shown in FIG. 9, is disposed in each of the first heating element receiving section 346 and the second heating element receiving section 376 in the connected position. The heating element 420 is therefore disposed between and in abutment with each of the first warming body 322 and the second warming body 352.

The warming base 380, as shown in FIGS. 4 and 5, is connected to a bottom of the connected first warming assembly 320 and second warming assembly 350 by a plurality of fasteners 388 extending through the fastener receiving passageways 384. In the shown embodiment, the fasteners 388 are screws. In other embodiments, the fasteners 388 may be a plurality of latches, an adhesive, or any other type of fastener known to those with ordinary skill in the art. As shown in FIG. 4, when the warming base 380 is connected to the first warming assembly 320 and the second warming assembly 350, the semi-circular sides 382 of the warming base 380 are complementary to and aligned with a bottom edge of the first outer wall 324 and a bottom edge of the second outer wall 354.

The cover housing 390, as shown in FIG. 6, is connected to a top of the connected first warming assembly 320 and second warming assembly 350 by a plurality of fasteners 378. In the shown embodiment, the fasteners 378 are screws. In other embodiments, the fasteners 378 may be a plurality of latches, an adhesive, or any other type of fastener known to those with ordinary skill in the art. The fasteners 378 are disposed in the column openings 336, 366 of the first fastener receiving columns 334 and the second fastener receiving columns 364 and fasten the fastener receiving columns 334, 364 to the cover fastener receiving passageways 399 inserted into the fastener receiving columns 334, 364 in the height direction H. In a position in which the cover housing 390 is connected to the top of the connected first warming assembly 320 and second warming assembly 350, as shown in FIG. 5, the first annular opening 393 corresponds to the second product receiving space 360 and the second annular opening 395 and outer cover recessed section 396 correspond to the first product receiving space 360 and the outer wall recessed section 332.

The inner housing 310 is disposed around the connected first warming assembly 320 and second warming assembly 350, forming a completely assembled temperature regulating device 300 shown in FIG. 4.

The assembled temperature regulating device 300 is disposed in the temperature regulating device receiving space 130 of the outer housing 100 as shown in FIGS. 1, 4, and 8. The cover housing 390 is complementary to the base housing 110. The temperature regulating device 300 is secured in the outer housing 100 by a plurality of fasteners 140 shown in FIGS. 4 and 9 extending through a bottom of the base housing 110 and engaging with the warming base 380.

The assembly of the heating system 400 within the product warming device 10 will now be described in greater detail.

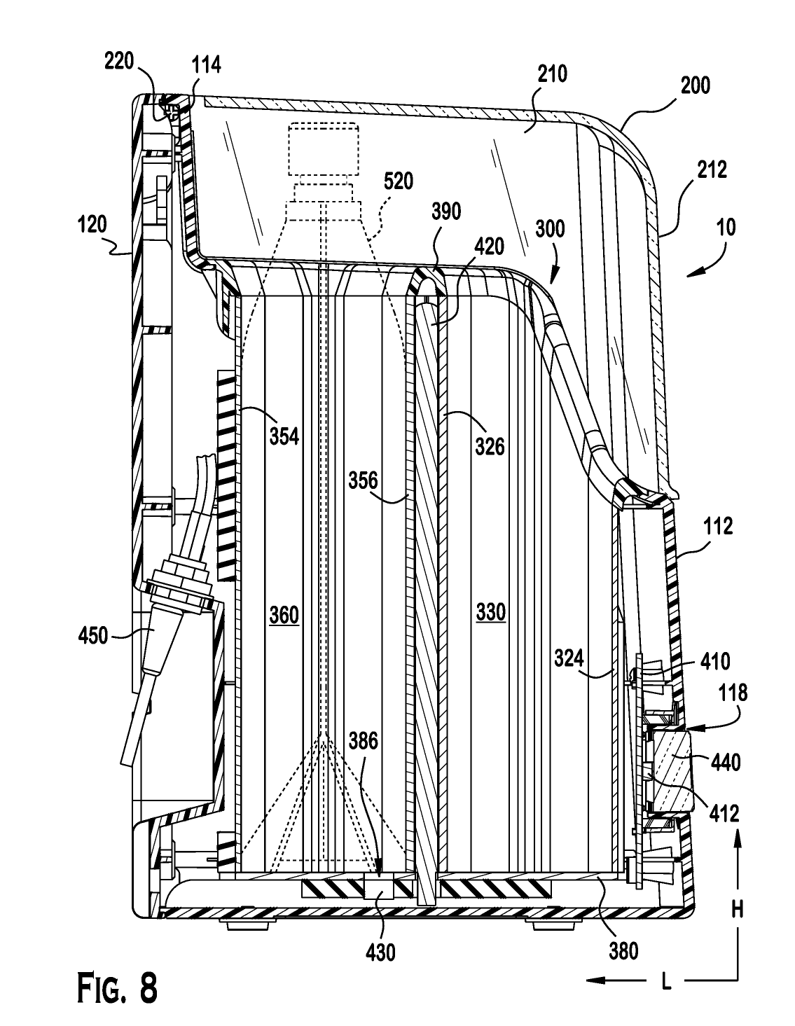

The heating element 420 of the heating system 400, as described above, is disposed between the first warming body 322 and the second warming body 352. As shown in FIG. 8, the controller 410 is attached to an inner portion of the front side 112 of the base housing 110 and is disposed between the first warming assembly 320 and the front side 112; the activation button 412 is disposed facing the actuation device receiving space 118. The actuation device 440 is disposed in the actuation device receiving space 118 and is movable within the actuation device receiving space 118 along the longitudinal direction L to move into and out of contact with the activation button 412. The temperature control device 430, as shown in FIGS. 5 and 8, is disposed in the temperature control device receiving space 386 of the warming base 380. The temperature control device 430 is thermally connected to the heating element 420 and the first warming body 322 and the second warming body 352. As shown in FIG. 8, a cord of the power supply 450 is inserted into the plug receiving passageway 122 of the back cover 120 in order to electrically connect to and provide power to the controller 410.

In the embodiment shown, the product warming device 10 is used to warm a plurality of containers 500 shown in FIG. 1. In an exemplary embodiment of the invention, the containers 500 include a first flexible pouch 510 smaller than a second flexible pouch 520. However, one skilled in the art should appreciate various containers, including rigid and flexible packages meant to retain a product. Furthermore, it should be noted, the product warming device 10 can be used to warm various product to be held inside of the container 500. For instance, foods, supplements, lotions, lubricants, and other known products can be heated within the product warming device 10 according to the invention.

The lid 200 is pivoted with respect to the base housing 110 to the open position shown in FIG. 1. The first flexible pouch 510 is inserted into the first package receiving space 330 and the second flexible pouch 520 is inserted into the second package receiving space 360 and, as shown in FIG. 2, the lid 200 is pivoted to the closed position. In other embodiments, a plurality of first flexible pouches 510 may be inserted into the first package receiving space 330. When inserted, the first flexible pouch 510 abuts the first warming body 322 and the second flexible pouch 520 abuts the second warming body 352.

To warm the containers 500, with the power supply 450 connected to an external power source and the lid 200 in the closed position shown in FIG. 2, a user depresses the actuation device 440 exposed through the actuation device receiving space 118 on the front side 112 of the base housing 110. Depressing the actuation device 440 moves the actuation device 440 along the longitudinal direction L shown in FIG. 8 into contact with the activation button 412, turning on the controller 410.

The controller 410 activates a warming mode when turned on. In the warming mode, the controller 410 receives an electrical signal from the temperature control device 430 indicative of a body temperature of the first and second warming bodies 322, 352 detected by the thermal connection described above. The memory of the controller 410 has a temperature set point stored thereon. The controller 410 compares the body temperature to the temperature set point. When the body temperature is below the temperature set point, the controller 410 sends an electrical signal to the temperature control device 430 to produce heat. The heat produced by the temperature control device 430 is thermally conducted into the heating element 420 and, via the heating element 420, into the first warming body 322 and the second warming body 352. The first warming body 322 and the second warming body 352 surrounding the containers 500 increase in temperature, evenly heating the containers 500 contained therein.

The controller 410 continuously receives the body temperature and, when the body temperature exceeds the temperature set point, the controller 410 sends an electrical signal to the temperature control device 430 to stop producing heat. The controller 410 controls the heat output by the temperature control device 430 to maintain the heating at the temperature set point and heat the containers to a corresponding desired temperature.

To remove the heated containers 500 from the product warming device 10, the user depresses the actuation device 440 to contact the activation button 412 and turn off the controller 410, deactivating the warming mode. The user then pivots the lid 200 from the closed position shown in FIG. 2 to the open position shown in FIG. 1 and manually removes the heated containers 500 from the first package receiving space 330 and the second package receiving space 360. Due to the larger opening to the first package receiving space 330 of the first warming assembly 320 formed by the outer wall recessed section 332 and the outer cover recessed section 396, it is easier for a user to access the smaller first flexible pouch 510 or plurality of first flexible pouches 510 for removal from the first package receiving space 330.

What is claimed is:

1. A product warming device, comprising:
an outer housing;
a temperature regulating device disposed within the outer housing and having:
a first warming assembly defining a first package receiving space and having, a first warming body with an outer wall recessed section expanding an upper opening of the first package receiving space; and
a second warming assembly defining a second package receiving space; and
a heating system disposed within the outer housing and adapted to heat the first warming assembly and the second warming assembly to a temperature setpoint.

2. The product warming device of claim 1, further comprising a lid pivotally attached to the outer housing.

3. The product warming device of claim 2, wherein the lid includes a transparent lid body and a hinge device rotatably connected to the outer housing.

4. The product warming device of claim 1, wherein the outer housing includes a base housing and a back cover.

5. The product warming device of claim 4, wherein the base housing includes a front side, a back side, and opposite sidewalls connecting the front side to the back side to define a temperature regulating device receiving space there between.

6. The product warming device of claim 5, wherein the base further includes an actuation device receiving space extend through the front side thereof.

7. The product warming device of claim 1, wherein the temperature regulating device includes an inner housing receiving the first warming assembly and the second warming assembly.

8. The product warming device of claim 7, wherein the first warming assembly and the second warming assembly are connected and both disposed within the inner housing.

9. The product warming device of claim 8, wherein the inner housing includes a front opening that extends continuously along three other perpendicular sides to define a warming assembly receiving space.

10. The product warming device of claim 9, wherein the inner housing tapers toward the front opening along height direction H.

11. The product warming device of claim 1, wherein the first warming assembly includes a first warming body having a first outer wall and a first inner wall connected by a pair of first warming sidewalls to define a first package receiving space.

12. The product warming device of claim 11, wherein the first outer wall and the first inner wall are both semi-circular shaped members having an elongated body.

13. The product warming device of claim 12, wherein the first outer wall includes an outer wall recessed section disposed on an upper edge thereof and expands an upper opening of the first package receiving space.

14. The product warming device of claim 12, wherein the first warming assembly further includes a first connection device disposed along the first inner wall 326 and having a first locking arm and a first guide arm connected to and extending away from the first inner wall.

15. The product warming device of claim 14, wherein the first locking arm and the first guide arm extend parallel to one another.

16. The product warming device of claim 15, further comprising a first latch disposed on an end of the first locking arm opposite the first inner wall and extends into a space between the first locking arm and the first guide arm.

17. The product warming device of claim 16, wherein the second warming assembly includes a second warming body having a second outer wall and a second inner wall connected by a pair of second warming sidewalls to define a second package receiving space.

18. The product warming device of claim 17, wherein the second outer wall further includes a second connection device disposed along the second inner wall and having a second locking arm and a second guide arm connected to and extending away from the second inner wall.

20. The product warming device of claim 19, wherein the second locking arm and the second guide arm extend parallel to one another and correspond with the first locking arm and the second guide arm.

21. The product warming device of claim 16, wherein the first warming assembly further includes a first heating element receiving section extending continuously along the first inner wall along a height H thereof.

22. The product warming device of claim 21, wherein the second warming assembly further includes a second heating element receiving section extending continuously along the second inner wall along a height H thereof, the second heating element receiving section corresponding with the first heating element receiving section.

23. The product warming device of claim 22, further comprising a second latch disposed on an end of the second locking arm opposite the second inner wall and extends into a space between the second locking arm and the second guide arm, the second latch engaging the first latch.

24. The product warming device of claim 22, wherein the heating system includes a heating element extending through the first heating element receiving section and the second heating element receiving section.

25. The product warming device of claim 24, wherein the heating element is connected to a temperature control device being controlled by a controller and is capable of generating heat in response to an electrical signal.

26. The product warming device of claim 25, wherein the temperature control device is a thermocouple and outputting an electrical signal indicative of a sensed temperature.
